(19) United States
(12) Patent Application Publication
(10) Pub. No.: US 2017/0354373 A1
(43) Pub. Date: Dec. 14, 2017

(54) SYSTEMS AND METHODS FOR DETECTING CARDIAC ACTIVITY AND/OR INACTIVITY

(71) Applicant: Stryker Corporation, Kalamazoo, MI (US)

(72) Inventor: Marko N. Kostic, Portage, MI (US)

(21) Appl. No.: 15/620,254

(22) Filed: Jun. 12, 2017

Related U.S. Application Data
(60) Provisional application No. 62/349,158, filed on Jun. 13, 2016.

(51) Int. Cl. A61B 5/00; A61B 5/0404; A61B 5/044; A61B 5/0408

(52) U.S. Cl. CPC A61B 5/6806; A61B 5/04085; A61B 5/0404; A61B 5/044; A61B 5/742; A61B 5/721; A61B 5/7415; A61B 2505/01; A61B 2562/0219

(57) ABSTRACT

A non-invasive sensor unit, such as a glove or pad, is adapted to be placed over the chest of an injured person who may be in need of CPR and/or AED. The sensor unit includes a plurality of electrodes for detecting one or more ECG voltages of the person. A controller processes the ECG voltages and displays them on a display, thereby enabling the user to assess whether CPR and/or AED is needed. The sensor unit is adapted to continue to provide ECG data while the user is applying chest compressions to the person. The sensor unit may include accelerometers for reducing artifacts from the ECG voltages that arise from the movement of the user while applying chest compressions. The electrodes are, in some embodiments, capacitive sensors that enable ECG voltages to be detected without requiring direct contact with the skin of the person.

### CROSS-REFERENCE TO RELATED APPLICATIONS

This application claims priority to U.S. provisional patent application Ser. No. 62/349,158 filed Jun. 13, 2016, by inventor Marko Kostic and entitled SYSTEMS AND METHODS FOR DETECTING CARDIAC ACTIVITY AND/OR INACTIVITY, the complete disclosure of which is incorporated herein by reference.

### BACKGROUND

The present disclosure relates to devices, systems, and methods for assisting a person prior to, during, and/or after performing cardio-pulmonary resuscitation (CPR) on a victim.

When performing CPR on a victim, it is typically necessary to occasionally pause during the application of chest compressions in order to determine whether the victim has reestablished his or her heart beat. Such pauses, however, interrupt the pumping of blood throughout the victim's body which is undesirable.

### SUMMARY

According to one or more embodiments, an apparatus and method for providing useful information about the cardiac state of the CPR-victim to the rescuer while the rescuer is performing CPR is provided. In some embodiments, the apparatus includes a glove worn by the rescuer that has a plurality of electrocardiograph (ECG) electrodes built into it. These electrodes provide the rescuer with information about the rhythmic activity, or lack of rhythmic activity, of the victim's heart. Further, this information is provided without requiring the rescuer to stop applying chest compressions. In some embodiments, the information is provided regardless of whether or not the glove makes direct contact with the skin of the victim, thereby enabling the rescuer to use the apparatus without requiring him or her to remove clothing from the victim's chest area.

According to one embodiment of the disclosure, a glove for use by a rescuer when performing CPR is provided. The glove includes a plurality of sheaths, first and second electrodes, and a controller. The sheaths are adapted to receive a plurality of digits (fingers and/or thumbs) of the rescuers hand. The first electrode is positioned adjacent a distal end of a first one of the sheaths and it is adapted to detect a first ECG voltage of the victim when the glove is worn by the rescuer and placed over the victim's chest. The second electrode is positioned away from the first electrode and it is adapted to detect a second ECG voltage of the victim when the glove is worn by the rescuer and placed over the victim's chest. The controller receives and processes the first and second ECG voltages and outputs a signal indicative of cardiac activity of the victim's heart based on the first and second ECG voltages.

According to another embodiment, a glove for use by a rescuer when performing CPR is provided. The glove includes a plurality of sheaths, first and second electrodes, a display, and a controller. The plurality of sheaths are adapted to receive a plurality of digits of the rescuer's hand. The first electrode is positioned adjacent a distal end of a first one of the sheaths and it is adapted to detect a first ECG voltage of the victim when the glove is worn by the rescuer and placed over the victim's chest. The second electrode is positioned away from the first electrode and it is adapted to detect a second ECG voltage of the victim when the glove is worn by the rescuer and placed over the victim's chest. The display is attached to a dorsal surface of the glove. The controller receives the first and second ECG voltages and displays data related to the first and second ECG voltages on the display.

According to yet another embodiment, a glove for use by a rescuer when performing CPR is provided. The glove includes at least four electrodes, an indicator, and a controller. The four or more electrodes are positioned on a ventral face of the glove at locations adapted to face the victim's chest when the glove is worn by the rescuer and used during chest compressions applied to the victim. The at least four electrodes are adapted to detect first, second, third, and fourth ECG voltages, respectively, when the glove is worn by the rescuer and placed over the victim's chest. The indicator is positioned on a dorsal face of the glove. The controller determines at least first, second, third and fourth signal-to-noise ratios (SNR) of the first, second, third, and fourth ECG voltages, respectively, and outputs a signal indicative of cardiac activity of the victim's heart based upon the ECG voltage with the highest SNR.

According to other aspects of the disclosure, the electrodes may be adapted to detect changes in capacitance due to electrical activity of the victim's heart. Such changes are detected due to the electrical activity of the victim's heart and are sensed by the electrodes without the electrodes needing to be placed in direct contact with the victim's skin.

In some embodiments, the second electrode is positioned on the glove at a location adjacent a palm of the rescuer's hand. In such embodiments, a third electrode may be placed on the glove at a location adjacent a distal end of a second one of the sheaths.

The indicator communicates with the controller and is adapted to provide at least one of an audio and visual indication to the rescuer of cardiac activity of the victim's heart. The indicator includes a graphical display, in some embodiments, that is adapted to display a graph derived from at least one of the first and second ECG voltages. In other embodiments, the indicator includes a Light Emitting Diode (LED) adapted to have its illumination state changed each time the victim's heart is detected to beat.

The glove further includes, in some embodiments, a wireless transceiver in communication with the controller. The controller uses the wireless transceiver to wirelessly transmit data regarding the first and second ECG voltages to a second device. In some of such embodiments, the controller wirelessly transmits data regarding the first and second ECG voltages to the second device while the rescuer is performing chest compressions on the victim.

In some embodiments, the glove includes a first accelerometer positioned adjacent the distal end of the first one of the sheaths and a second accelerometer positioned adjacent the distal end of a second one of the sheaths. The controller uses outputs from the first and second accelerometers to remove artifacts from the first and second ECG voltages that are due to movement of the rescuer when applying chest compressions to the victim.

The glove includes a first dielectric layer positioned between the first electrode and an interior of the first one of the sheaths, in some embodiments. The first dielectric layer shields the first electrode from electrical interference caused by the rescuers hand.

In some embodiments, the controller displays a difference between the first and second ECG voltages on the display. The difference may be displayed on a graph with respect to an axis indicating time.

The display, in at least one embodiment, is a liquid crystal display, an AMOLED display, and/or an OLED display.

The controller, in some embodiments, outputs at least two signals indicative of cardiac activity of the victim's heart based upon the ECG voltages with the first and second highest SNRs. The controller graphically displays data derived from the voltages having the first and second highest SNRs.

In some embodiments, the glove includes at least four accelerometers. Each of the accelerometers is positioned adjacent a corresponding one of the electrodes. The controller uses outputs from the at least four accelerometers to remove artifacts from the first, second, third, and fourth ECG voltages that are due to movement of the rescuer when applying chest compressions to the victim. The controller may remove the artifacts from the first, second, third, and fourth ECG voltages prior to determining the ECG voltage with the highest SNR.

A first one of the electrodes is positioned adjacent a lower palm area, in some embodiments, and a second one of the electrodes is positioned adjacent a first fingertip of the glove. A third electrode may be positioned adjacent a second fingertip of the glove.

In some embodiments, one or more of the accelerometers are used to detect a depth of the chest compressions and provide an indication of the depth to the rescuer.

In those embodiments where a display is included on the glove, the display may be positioned outside of an opisthenar portion of the glove such that the rescuer is able to place his or her second hand (which may be gloved or non-gloved) over the opisthenar portion of the glove on the first hand while applying chest compressions. In one such embodiment, the display is positioned adjacent a wrist area of the glove.

In yet another embodiment, a system is provided for use by a rescuer when performing CPR compressions on a victim. The system includes first and second gloves. The first glove has a plurality of sheaths adapted to receive a plurality of digits of the rescuer's hand; first and second electrodes adapted to detect first and second ECG voltages, respectively, when the first glove is worn by the rescuer and placed over the victim's chest; and a first transceiver. The second glove has a second transceiver and a display attached to a dorsal surface of the glove. The second transceiver is adapted to receive data related to the first and second ECG voltages from the first transceiver and to use the received data to display ECG information about the victim on the display.

The first and second transceivers are adapted to communicate via a wire in one embodiment, and wirelessly in another embodiment.

In still another embodiment, a method of performing CPR on a victim is provided. The method includes placing a plurality of ECG electrodes over the victim's chest wherein the plurality of ECG electrodes produce a corresponding plurality of ECG voltages. The method further includes placing a first hand over at least one of the ECG electrodes; placing a second hand over the first hand; applying compressive forces to the victim's chest; determining which of the plurality of ECG voltages has the best signal-to-noise (SNR) ratio; and outputting a signal indicative of cardiac activity of the victim's heart based on the ECG voltage with the best SNR.

The plurality of ECG electrodes are incorporated into a glove, in a first embodiment. In another embodiment, the plurality of ECG electrodes are incorporated into a pad placed over the victim's chest.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a pair of gloves according to a first embodiment of the disclosure;

FIG. 2 is a side elevation view of a left one of the gloves of FIG. 1;

FIG. 3 is a block diagram of a control system for the gloves of FIG. 1;

FIG. 4 is a block diagram of an alternative control system for one or more modified gloves according to a second embodiment of the disclosure; and

FIG. 5 is a block diagram of yet another alternative control system for one or more modified gloves according to a third embodiment of the disclosure.

### DETAILED DESCRIPTION OF THE EMBODIMENTS

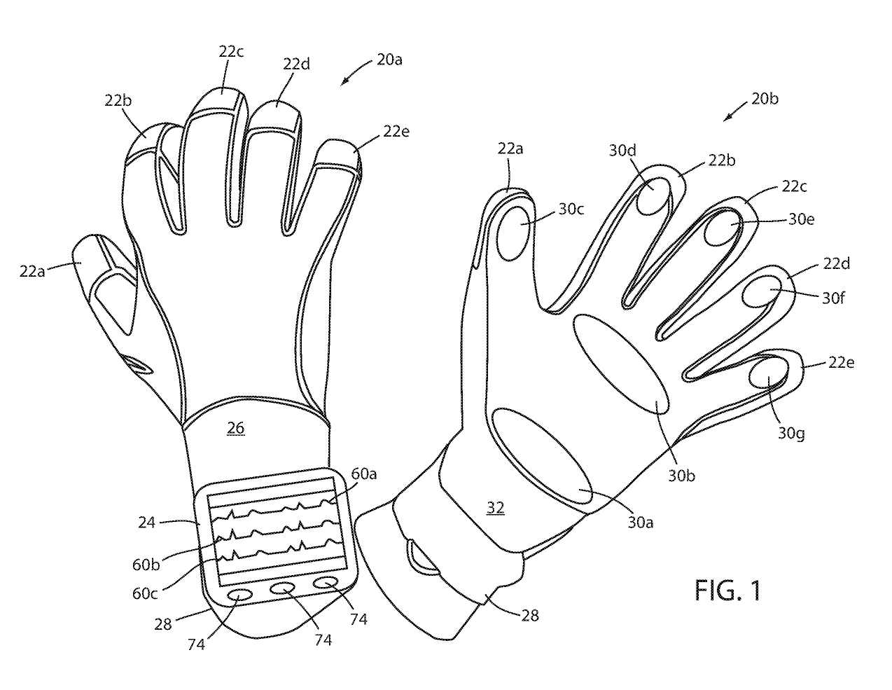

A first glove 20a and a second glove 20b according to a first embodiment of the disclosure are shown in FIG. 1. First and second gloves 20a and 20b are each constructed in the same manner in this embodiment. First glove 20a is adapted to be worn on the right hand of a rescuer while second glove 20b is adapted to be worn on the left hand of the rescuer. Each glove 20a and 20b includes five sheaths 22a-e that are each adapted to receive one of the rescuers digits (fingers and/or thumb). More specifically, sheaths 22a are adapted to receive the rescuer's thumbs; sheaths 22b are adapted to receive the rescuer's index fingers; sheaths 22c are adapted to receive the rescuers middle fingers; sheaths 22d are adapted to receive the user's ring fingers, and sheaths 22e are adapted to receive the user's pinky fingers.

Gloves 20a and 20b are intended to be worn by a rescuer while the rescuer is performing chest compressions on a victim whose heart has stopped beating. Gloves 20a and 20b are adapted to detect ECG activity of the victim's heart while the rescuer is performing chest compressions and to display one or more signals indicative of that activity (or inactivity as the case may be). More specifically, each glove 20a and 20b includes a display 24 coupled to a dorsal side 26 of the gloves 20a and 20b and positioned in a wrist area 28. Display 24 displays information relating to the ECG voltages detected by the glove in a manner that allows the rescuer to see the displayed information while simultaneously applying chest compressions. The rescuer is therefore able to continue applying chest compressions without interruption while simultaneously receiving an indication of whether or not the victim's heart has regained any rhythmic activity or not. This avoids the need for the rescuer to periodically stop the chest compressions and manually check for cardiac activity, thereby also avoiding the cessation of blood being pumped to the victim's vital organs by way of the applied chest compressions.

Each glove 20a, 20b includes a plurality of electrodes 30 positioned on a ventral side 32 of the gloves. In the illustrated embodiment, each glove 20a, 20b includes a lower palm electrode 30a, an upper palm electrode 30b, a thumb electrode 30c, an index finger electrode 30d, a middle finger electrode 30e, a ring finger electrode 30f, and a pinky finger electrode 30g. The electrodes 30c-30g are each placed adjacent the distal ends of the respective sheaths 22 to which they are attached. It will be understood by those skilled in the art that more or fewer numbers of electrodes 30 may be used in alternative embodiments, and that the position of one or more of the electrodes 30 may also be changed from that illustrated in FIG. 1.

Each electrode 30 is made of a suitable metal and may be implemented either as a conductive electrode or a capacitive electrode. When implemented as conductive electrodes, gloves 20a and/or 20b are most effective at detecting cardiac voltages when the electrodes 30 are placed in direct contact with the victim's skin. When implemented as capacitive electrodes, gloves 20a and/or 20b are able to detect cardiac voltages without having to place the electrodes 30 in direct contact with the victim's skin. In this latter implementation, gloves 20a and 20b are therefore able to effectively operate without requiring the rescuer to remove clothing from the victim's chest area.

Electrodes 30, whether implemented as resistive or capacitive electrodes, detect changes in electrical potential on or near the victim's skin due to the cardiac activity of the victim. These changes in electrical potential are sensed when the electrodes 30 positioned in contact with the victim's skin (if implemented as resistive electrodes), or when the electrodes 30 are positioned sufficiently close to the victim's heart so as to detect changes in electrical potential due to cardiac activity of the victim's heart (if implemented as capacitive electrodes). When one of gloves 20a or 20b is placed over the victim's chest and includes capacitive electrodes 30, the electrodes 30 are sufficiently close to the victim's heart to sense such electrical potential changes, even if the victim still has clothing positioned on top of his or her chest and located between electrodes 30 and the victim's skin.

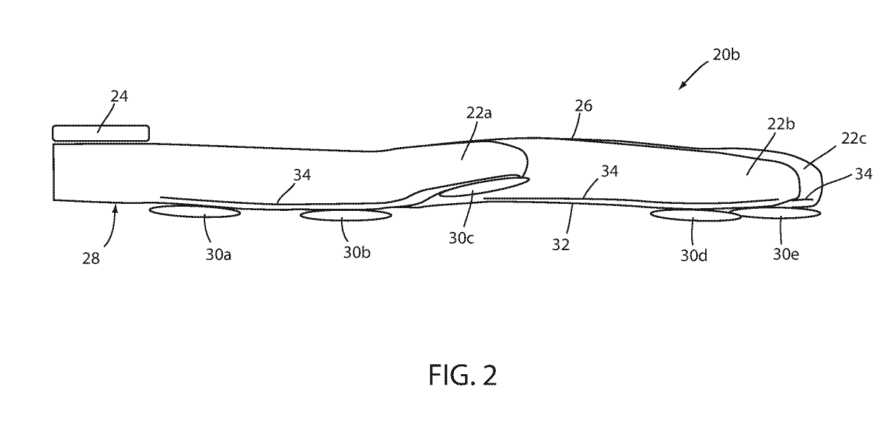

When electrodes 30 are implemented as capacitive sensors, gloves 20a, 20b are constructed, in at least one embodiment, to include a dielectric insulation layer 34 positioned between the electrodes 30 and the interior of the gloves 20a, 20b, as shown in FIG. 2. Dielectric insulation layer 34 helps prevent changes being detected by electrodes 30 due to the pulse of the rescuer and/or physical movement of the rescuer's hands and/or fingers. Dielectric insulating layers 34 are positioned inside of each sheath 22, as well as over a palm area of the gloves 20a, 20b such that each of the electrodes 30 is shielded from electrical activity of the rescuers hands.

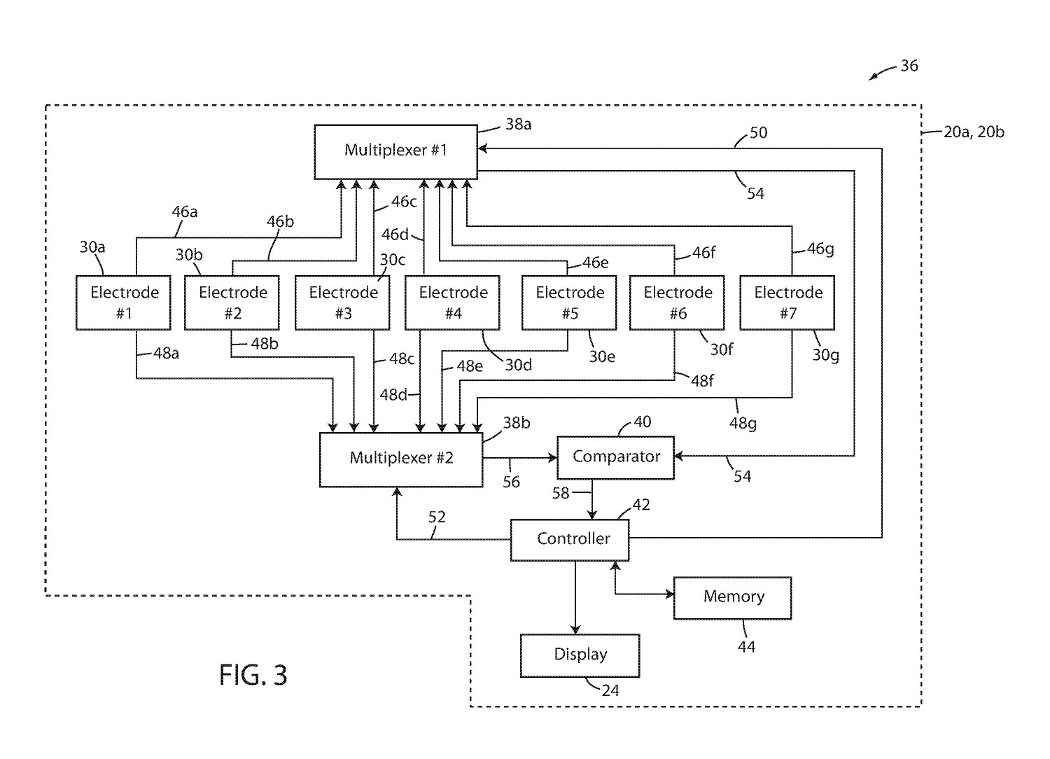

One example of a control system 36 of gloves 20a, 20b is shown in diagram form in FIG. 3. Control system 36 includes electrodes 30a-g, first and second multiplexers 38a, 38b, a comparator 40, a controller 42, a memory 44, and display 24. Control system 36 controls and carries out the detection, processing, and display of the voltages sensed by electrodes 30. Controller 42 of control system 36 is a microcontroller in one embodiment of control system 36. Controller 42 may alternatively be constructed of any electrical component, or group of electrical components, that are capable of carrying out the functions described herein. In other embodiments, controller 42 includes any one or more microprocessors, microcontrollers, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controller 42 in carrying out the functions described herein, as well as the data necessary for carrying out these functions are stored in memory 44, which is accessible to controller 42.

Electrodes 30a-g are coupled to first multiplexer 38a by first input lines 46a-g, respectively. Electrodes 30a-g are also coupled to second multiplexer 38b by second input lines 48a-g, respectively. The electrical potentials or voltages from each of electrodes 30a-g are therefore fed to both multiplexers 38a and 38b. Controller 42 is electrically coupled to first and second select lines 50 and 52. First select line 50, which is controlled by controller 42, is coupled to first multiplexer 38a while second select line 52 is coupled to second multiplexer 38b. First select line 50 controls which one of input lines 46a-g multiplexer 38a outputs onto first output line 54. Second select line 52 controls which one of input lines 46a-g multiplexer 38b outputs onto second output line 56. First and second output lines 54 and 56 feed into comparator 40. Comparator 40 includes an output line 58 that feeds back into controller 42.

Controller 42 is programmed to use comparator 40 and multiplexers 38a, 38b to detect and record voltage differences between pairs of electrodes 30a-g. In one embodiment, controller 42 is programmed to output a control signal on first select line 50 that selects the input line 46a from first electrode 30a. This electrode 30a corresponds to the lower palm area of the rescuers hand. The result of this selection is to feed the output from electrode 30a to comparator 40. Controller 42 is also programmed to output a control signal on second select line 52 that selects the input line 46d from fourth electrode 30d. This electrode 30d corresponds to the index finger of the rescuer's hand. The result of this latter selection is to feed the output from electrode 30d to comparator 40. Comparator 40 therefore compares the outputs from first and fourth electrodes 30a and 30d and feeds the result of the comparison to controller 42 via output line 58. Controller 42 processes the result by determining a magnitude of the difference between the two voltages from electrodes 30a and 30d. This difference is stored in memory 44.

After detecting and measuring the difference in voltages from electrodes 30a and 30d, controller 42 changes the values output on select lines 50 and 52 such that a comparison is made by comparator 40 between one or more other electrodes (for example, electrodes 30a and 30e). The result of this comparison is reported by comparator 40 to controller 42 via output line 58, which measures the difference and stores the value. Controller 42 may then carry out one or more additional comparisons in a similar manner, including measuring and storing the difference in voltages resulting from the additional comparisons. As would be known to one of ordinary skill in the art, additional processing of the voltages detected by electrodes 30 may also be carried out by controller 42 to generate meaningful ECG voltages for display on display 24. Such additional processing may include appropriate filtering and analysis of the raw voltages coming from electrodes 30, as would be known to one of ordinary skill in the art. The processed data output by controller 42 may indicate the victim's heart rate (if any), the presence of arrhythmia, and/or other cardiac data.

After comparing and processing the voltages from at least two pairs of electrodes 30, controller 42 is programmed to select the pair of electrodes having the greatest difference between their respective voltages. Controller 42 thereafter displays the outputs from that pair of voltages on display 24.

The output from that pair of voltages is indicative of the electrical activity of the victim's heart (if any), and provides a signal to the rescuer as to whether or not the victim's heart has started beating again.

In some embodiments, controller 42 is programmed to display multiple voltages. The multiple voltages are generated from the differences between different pairs of electrodes 30. For example, in the embodiment shown in FIG. 1, controller 42 is programmed to display three different output voltages 60a, 60b, and 60c. The selection of which electrode pairs are used to generate these three different output voltages 60a-60c is preprogrammed and fixed, in some embodiments. In other embodiments, the selection of the electrode pairs that are used to generate these three different output voltages 60a-60c is preprogrammed, but may be changed by the user utilizing one or more buttons (e.g. buttons 74) coupled to display 24. In still other embodiments, the selection of the electrode pairs that are used to generate these different output voltages 60a-60c is dynamic. When programmed to be dynamic, controller 42 may run comparisons between multiple pairs of electrodes 30 and choose the three pairs having the highest differences. Alternatively, when programmed to be dynamic, controller 42 may run comparisons between multiple pairs of electrodes 30 and choose the pairs having the three highest signal-to-noise (SNR) ratios. Still other methods of choosing which voltages to display are possible. Further, the number of voltages 60 displayed on display 24 may vary from that shown in FIG. 1.

The ECG output voltages that are displayed on display 24 need not be of the type that are clinically accurate enough to provide detailed cardiac information, such as one might find from a standard 12-lead electrocardiogram. Instead, the ECG voltages that are displayed on display 24 need only provide an indication of cardiac activity or inactivity. In some embodiments, the output ECG voltages will only provide an indication of the QRS complex of the victim's cardiac activity. In other embodiments, the output ECG voltages will provide an indication of the PQRST complex of the victim's cardiac activity. Indeed, in some embodiments, display 24 is replaced by a non-graphical indicator that merely provides a flashing light (such as an LED) and/or a beeping sound whenever a heartbeat is detected by control system 36. In such embodiments, control system 36 doesn't provide any morphological data about the ECG voltages, but instead merely provides an indication whenever a heartbeat is detected. This allows the rescuer to determine not only whether the victim's heart is beating or not, but also the frequency of the heart beats. Further, in some embodiments, the brightness of the visual indicator and/or the volume of the aural indicator may be tied to the strength of the detected heart beat so that the rescuer is given feedback as to the strength of the victim's cardiac activity.

Due to the fact that gloves 20a and/or 20b are intended, in some embodiments, to provide indications of the presence or absence of cardiac activity, but not necessarily details about the morphology of the ECG waves, it is not necessary for a rescuer to place his or her hand that contacts the victim in a specific orientation or location with respect to the victim. That is, the electrodes 30 do not need to align with any of the locations that electrodes are conventionally placed on the victim's body when a standard 12-lead electrocardiogram is obtained. The rescuer therefore does not need to be concerned with proper position or orientation while using gloves 20a, 20b, but can instead focus his or her energies on applying chest compressions.

Regardless of the manner in which control system 36 conveys the ECG data to the rescuer (e.g. via display 24 or one or more non-graphical indicators), control system 36 repetitively analyzes the outputs of electrode pairs multiple times a second. Control system 36 is therefore able to detect cardiac activity in real time and to output, as appropriate, voltages corresponding to the detected cardiac activity in real time, using either display 24 or one or more non-graphical indicators.

It will be understood by those skilled in the art that the circuitry of control system 36 may be varied substantially from that shown in FIG. 3. For example, although not shown in FIG. 3, it will be understood by those skilled in the art that one or more analog-to-digital converters (ADC) may be added to the circuitry for converting the analog outputs from electrodes 30 into digital signals for processing by controller 42. As another example, additional circuitry and/or programming of controller 42 may be added that looks for signal-to-noise (SNR) ratios between pairs of electrodes 30. In some of such embodiments, controller 42 displays the ECG voltages that are generated from the pair of electrodes 30 having the highest SNR. In other of such embodiments, controller 42 displays the ECG voltages that are generated from several of the pairs of electrodes having the highest SNRs. In still other embodiments, the outputs from one or more pairs of electrodes are combined together with the outputs from one or more other pairs of electrodes 30. Still other manners may be used for detecting ECG voltages from electrodes 30.

As shown in FIG. 1, display 24 is positioned outside of an opisthenar region (the back side of the palm) of the gloves 20a, 20b. This allows the rescuer to place a first one of his or her hands in direct contact with the victim and then place the second one of or his or her hands directly on top of the opisthenar region of the first hand in the standard manner recommended for performing chest compressions. Because displays 24 are positioned outside of this opisthenar region, the placement of the second hand on top of the first hand does not visually obscure the display coupled to the glove 20a, 20b of the first hand, thereby enabling the rescuer to view the presence or absence of cardiac activity while maintaining his or her hands on top of each other and applying chest compressions. In other words, the placement of display 24 on gloves 20a, 20b of the embodiment of FIG. 1 enables the display to be seen while the rescuer performs CPR in the conventional manner with one hand on top of the other.

In a modified embodiment, only a single one of the gloves 20a, 20b includes a display 24. In this modified embodiment, the glove with the display 24 is first positioned by the rescuer on top of the victim's chest and the glove without the display is then placed on top of the glove with the display. In this manner, the glove with the display has its electrodes placed in contact with the victim, while the glove without the display is not necessarily making any contact between its electrodes and the victim. Indeed, in this embodiment, the glove without the display does not need to include any electrodes 30 at all as it does not include any structure for displaying the output of the voltages sensed by electrodes 30. The glove without the display 24 therefore may omit all of the control system 36 components whatsoever, if desired. Alternatively, the rescuer can omit using a second glove and can instead place his or her gloved hand in contact with the victim and then place his or her non-gloved hand on top of the gloved hand. Thereafter, chest compressions can be applied using both hands.

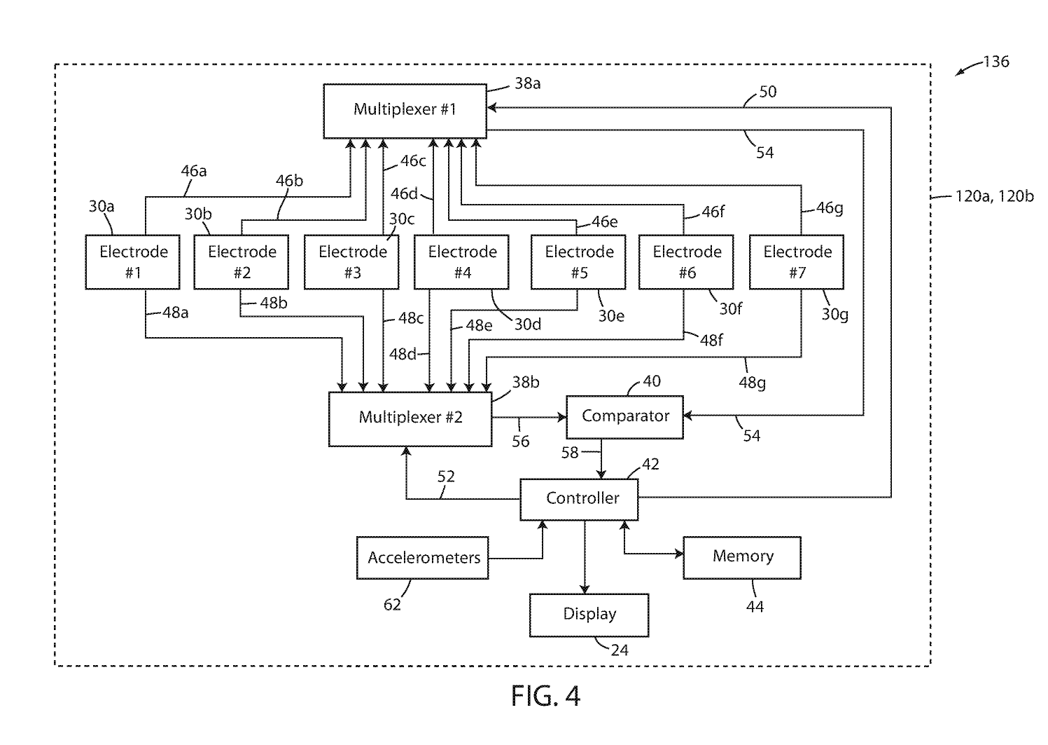

FIG. 4 illustrates a modified control system 136 that may be used in one or more modified gloves 120a and/or 120b. Modified control system 136 includes a plurality of components that are the same as the components of control system 36. Those common components are labeled with the same reference number as used with control system 36. New components are labeled with a new reference number. Further, unless explicitly stated otherwise below, those components of control system 136 that are common to control system 36 operate in the same manner as previously described.

Control system 136 differs from control system 36 in that it also includes one or more accelerometers 62. Control system 136 includes, in at least one embodiment, at least one accelerometer for each digit of the rescuers hand. In other embodiments, fewer or greater numbers of accelerometers 62 may be used. Regardless of the specific number of accelerometers included within control system 136, each accelerometer 62 is coupled to controller 42 and outputs signals indicative of the acceleration of portions of gloves 120a, 120b. Controller 42 uses these signals to reduce artifacts in the ECG voltages from electrodes 30 that may be otherwise introduced due to the movement of the rescuers hands during chest compressions. That is, accelerometers 62 sense the acceleration of the rescuer's hands and/or fingers while the rescuer is applying chest compressions to the victim. These outputs are forwarded to controller 42, which is programmed to use these outputs, to filter disturbances in the ECG voltages that arise from the movement of the rescuer's hands. In this manner, the presence of ECG voltages is not falsely reported due to electrical disturbances caused by the rescuers hand motion. The accuracy of the reported ECG voltages is also increased.

Controller 42 of control system 136 may also be programmed, in one embodiment, to additionally detect the depth of the rescuer's chest compressions using the outputs of accelerometers 62. In one of these modified embodiments, controller 42 displays the depth on display 24. In another of these modified embodiments, controller 42 displays a target depth simultaneously with the actually measured depth so that the rescuer can visually see if he or she is under-compressing or over-compressing the victim's chest. In still another one of these modified embodiments, controller 42 provides an aural signal indicative of the chest compression depth, either in addition to, or in lieu of, the visual display of the chest compression depth. The aural signal may comprise a sound of varying pitch wherein a first pitch corresponds to compressions that are too shallow, a second pitch corresponds to compressions that are too deep, and a third pitch corresponds to compressions that are acceptable.

In still another modified embodiment, controller 42 only uses the outputs of accelerometers 62 for detecting the depth of the applied compressions, and does not use accelerometers 62 for removing artifacts from the ECG voltages.

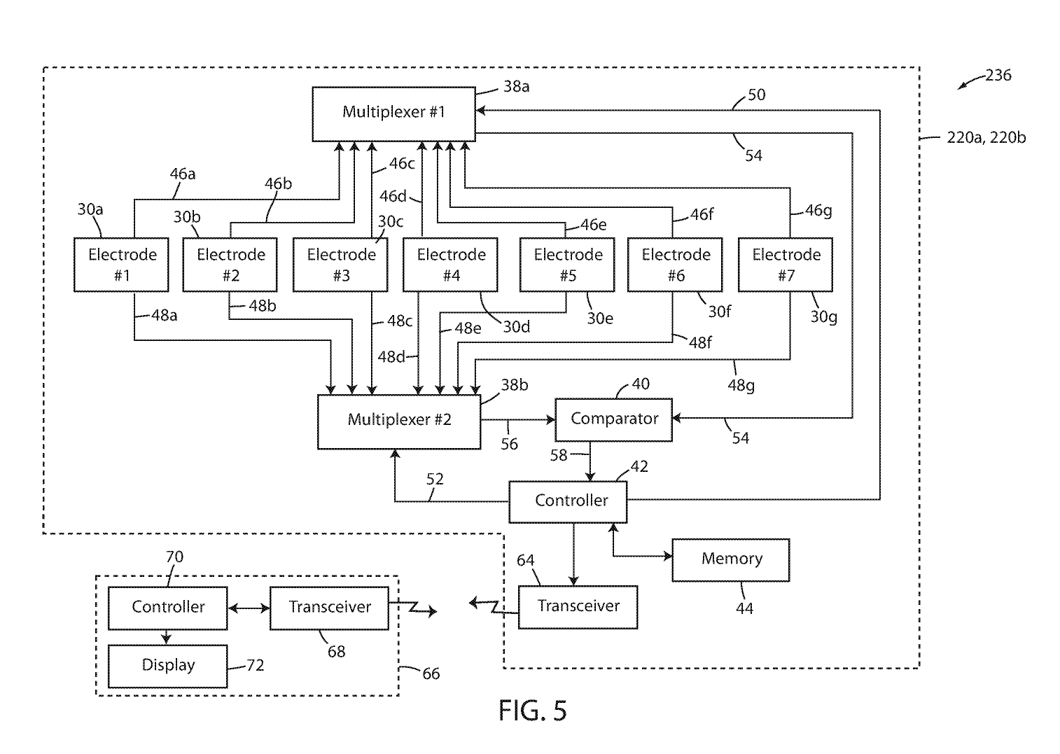

FIG. 5 illustrates a second modified control system 236 that may be used in one or more modified gloves 220a and/or 220b. Modified control system 236 includes a plurality of components that are the same as the components of control system 36. Those common components are labeled with the same reference number as used with control system 36. New components are labeled with a new reference number. Further, unless explicitly stated otherwise below, those components of control system 236 that are common to control system 36 operate in the same manner as previously described.

Control system 236 differs from control systems 36 and 136 in that it includes a transceiver 64 instead of a display 24. Control system 236 operates in the same manner as control system 36 except that instead of displaying one or more ECG waveforms and/or other data on display 24, transceiver 64 transmits the data that would otherwise be displayed to another device 66. In one embodiment, transceiver 64 is a Bluetooth transceiver (e.g. IEEE 8020.15.1). In other embodiments, transceiver 64 is a WiFi transceiver (e.g. IEEE 802.11) or a ZigBee transmitter (e.g. IEEE 802.15.4). In still other embodiments, transceiver 64 is a wired transceiver, such as, but not limited to, a Universal Serial Bus (USB) transceiver.

The other device 66 may vary. In some embodiments, the other device 66 is any one of a defibrillator/monitor (such as, but not limited to, a LifePak defibrillator marketed by Physio-Control of Redmond, Washington), a smart phone, a wristband monitor, a tablet, a personal computer, a server, a pair of smart glasses (e.g. Google Glass), a network, or a smart watch, such as the iWatch marketed by Apple Inc. of Cupertino, Calif. The other device 66 includes a transceiver 68, a controller 70, and a display 72. Transceiver 68 is adapted to communicate with transceiver 64. Controller 70 may be a microcontroller, microprocessor, or other set of circuitry capable of processing the data received from transceiver 68 and displaying it on display 72. Display 72 may be the same type of display as display 24, or it may be different.

When the other device 66 is a watch worn by the rescuer, or a nearby tablet, smart phone, or other device, the rescuer can see the cardiac rhythm information displayed on the watch, tablet, or other device, while simultaneously performing chest compressions. The rescuer therefore does not need to stop the chest compressions to assess the cardiac status of the victim.

It will be understood by those skilled in the art that, although control system 236 does not include any accelerometers 62, it could be modified to include one or more accelerometers in order to perform either or both of the previously described functions: (1) removing motion-induced artifacts from the ECG voltages; and/or (2) measuring the depth of the chest compressions and reporting the depth in a meaningful manner to the rescuer.

It will also be understood by those skilled in the art that control system 236 may be additionally or alternatively modified to include both a display 24 and transceiver 64. By including both display 24 and transceiver 64, the glove 220a, 220b is able to display the ECG data locally as well as transmit it to the other device 66. The other device 66 may also display the data and/or it may store the data in a memory (not shown).

Still further, it will be understood that in another modified embodiment, a first one of gloves 220a and 220b includes transceiver 64 but no display 24, while a second one of gloves 220a and 220b includes both a transceiver 64 and a display 24. In this modified embodiment, if the rescuer places the first glove on the victim and positions the second glove on top of the first glove, the first glove transmits the ECG data to the second glove for display on display 24. On the other hand, if the rescuer places the second glove on the victim and positions the first glove on top of the second glove, the second glove displays the ECG data on its display. In this embodiment, the pair of gloves 220a and 220b need only include a single display. Further, regardless of which order the rescuer stacks his or her hands on top of the victim's chest, the ECG data will be automatically displayed on the display 24 of the one glove that includes display 24.

In still other embodiments, any of the control systems 36, 136, and/or 236 disclosed herein may be incorporated into a pad that, instead of being worn by the rescuer, is placed on top of the victim's chest in the area of the victim's heart. The rescuer then pushes down on the pad while applying chest compressions to the victim. A display is either coupled to the pad or attached to another device 66 that receives the ECG data from the pad. In these pad embodiments, the rescuer does not need to put on any gloves when applying chest compressions to the victim, but still is able to receive information about the presence of absence of the victim's cardiac activity while applying the chest compressions.

As yet another alternative embodiment, either the pads or any of the gloves 20, 120, and/or 220 may be modified to include one or more additional sensors. For example, in some embodiments, one of these pads and/or gloves includes a saturated oxygen sensor that reports its data on display 24 and/or display 72. One such saturated oxygen sensor that may be combined with gloves 20, 120, 220, and/or the pads is disclosed in commonly assigned U.S. patent publication 2015/0327777, filed by inventors Marko Kostic, et al. and entitled TISSUE MONITORING APPARATUS AND SYSTEM, the complete disclosure of which is incorporated herein by reference. Other types of saturated oxygen sensors may also be used.

Another type of sensor that may be incorporated into any of the gloves 20, 120, 220, and/or pads is a pulse wave velocity sensor. One such pulse wave velocity sensor suitable for this purpose is disclosed in commonly assigned U.S. patent publication 2016/0120411, filed by inventors Sean Hadley et al. and entitled SYSTEMS AND METHODS FOR DETECTING PULSE WAVE VELOCITY, the complete disclosure of which is incorporated herein by reference. Other types of pulse wave velocity sensors may also be used. Regardless of the type of pulse wave velocity sensor utilized, the processed outputs from the sensor are displayed on displays 24 and/or 72.

Although gloves 20, 120, and 220, as well as the pads, have all been described herein as being used for providing cardiac information to the rescuer while the rescuer is applying chest compressions, it will be understood by those skilled in the art that these devices may alternatively or additionally be used to provide a quick assessment of the victim's cardiac activity either before or after applying chest compressions. For example, one of these devices may be placed over the victim's chest prior to performing chest compressions to first determine whether CPR or AED (automated external defibrillator) treatments are needed or not. Also, if the chest compressions, or other treatment, are successful at restarting the victim's heart, the gloves or pads may be used to thereafter monitor the victim's cardiac rhythms.

Either of both of displays 24 and 72 are Liquid Crystal Displays (LCD) in one embodiment. In another embodiment, either or both of displays 24 and 72 are Active Matrix Organic Light Emitting Diode (AMOLED) displays. In still another embodiment, either or both of displays 24 and 72 are Organic Light Emitting Diode (OLED) displays. Still other display technology may incorporated into displays 24 and/or 72, such as, but not limited to, Organic Light Emitting Transistor (OLET) displays, Surface-Conduction Electron-Emitter (SED) displays, Field Emission (FED) displays, Quantum Dot (QD-LED) displays, Ferro Liquid Crystal Displays (FLCD), and/or Thick-Film Dielectric Electroluminescent Technology (TDEL) displays.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A glove for use by a rescuer when performing Cardio Pulmonary Resuscitation (CPR) compressions on a victim, the glove comprising:
a plurality of sheaths adapted to receive a plurality of digits of the rescuer's hand;
a first electrode positioned adjacent a distal end of a first one of the sheaths, the first electrode adapted to detect a first ECG voltage of the victim when the glove is worn by the rescuer and placed over the victim's chest;
a second electrode positioned away from the first electrode, the second electrode adapted to detect a second ECG voltage of the victim when the glove is worn by the rescuer and placed over the victim's chest; and
a controller adapted to receive and process the first and second ECG voltages, the controller adapted to output a signal indicative of cardiac activity of the victim's heart based on the first and second ECG voltages.

2. The glove of claim 1 further comprising:
a third electrode positioned away from the first and second electrodes, the third electrode adapted to detect a third ECG voltage of the victim when the glove is worn by the rescuer and placed over the victim's chest; and
wherein the controller is further adapted to receive the third ECG voltage and to process the third ECG voltage in conjunction with at least one of the first and second ECG voltages when outputting the signal indicative of cardiac activity of the victim's heart.

3. The glove of claim 1 wherein the first and second electrodes are adapted to detect changes in capacitance due to electrical activity of the victim's heart.

4. The glove of claim 3 wherein the first and second electrodes detect changes in capacitance due to the electrical activity of the victim's heart without needing to be placed in direct contact with the victim's skin.

5. The glove of claim 1 wherein the second electrode is positioned on the glove at a location adjacent a palm of the rescuers hand.

6. The glove of claim 2 wherein the second electrode is positioned on the glove at a location adjacent a palm of the rescuers hand, and the third electrode is positioned on the glove at a location adjacent a distal end of a second one of the sheaths.

7. The glove of claim 1 further comprising an indicator attached to a dorsal surface of the glove, the indicator in communication with the controller and adapted to provide at least one of an audio and visual indication to the rescuer of cardiac activity of the victim's heart.

8. The glove of claim 1 further comprising a wireless transceiver in communication with the controller, the controller adapted to use the wireless transceiver to wirelessly transmit data regarding the first and second ECG voltages to a second device.

9. The glove of claim 8 wherein the controller is adapted to wirelessly transmit data regarding the first and second ECG voltages to the second device while the rescuer is performing chest compressions on the victim.

10. The glove of claim 1 further comprising:
a first accelerometer positioned adjacent the distal end of the first one of the sheaths;
a second accelerometer positioned adjacent the distal end of a second one of the sheaths; and
wherein the controller is further adapted to use outputs from the first and second accelerometers to remove artifacts from the first and second ECG voltages that are due to movement of the rescuer when applying chest compressions to the victim.

11. The glove of claim 3 further including a first dielectric layer positioned between the first electrode and an interior of the first one of the sheaths, the first dielectric layer adapted to shield the first electrode from electrical interference caused by the rescuer's hand.

12. The glove of claim 2 wherein the controller is further adapted to determine first, second, and third signal-to-noise ratios (SNR) of the first, second, and third ECG voltages, respectively, compare the ECG voltage with the highest SNR to the ECG voltage with the second highest SNR, and to output the signal indicative of cardiac activity of the victim's heart based on the comparison.

13. A glove for use by a rescuer when performing Cardio Pulmonary Resuscitation (CPR) compressions on a victim, the glove comprising:
a plurality of sheaths adapted to receive a plurality of digits of the rescuer's hand;
a first electrode positioned adjacent a distal end of a first one of the sheaths, the first electrode adapted to detect a first ECG voltage of the victim when the glove is worn by the rescuer and placed over the victim's chest;
a second electrode positioned away from the first electrode, the second electrode adapted to detect a second ECG voltage of the victim when the glove is worn by the rescuer and placed over the victim's chest;
a display attached to a dorsal surface of the glove; and
a controller adapted to receive the first and second ECG voltages and to display data related to the first and second ECG voltages on the display.

14. The glove of claim 13 wherein the controller is adapted to display a difference between the first and second ECG voltages on the display.

15. The glove of claim 14 wherein the controller displays a graph of the difference between the first and second ECG voltages with respect to a time axis on the display.

16. The glove of claim 13 further comprising:
a first accelerometer positioned adjacent the distal end of the first one of the sheaths;
a second accelerometer positioned adjacent the distal end of a second one of the sheaths; and
wherein the controller is further adapted to use outputs from the first and second accelerometers to remove artifacts from the first and second ECG voltages that are due to movement of the rescuer when applying chest compressions to the victim, the controller removing the artifacts prior to displaying the data related to the first and second ECG voltages on the display.

17. The glove of claim 13 wherein the first and second electrodes are adapted to detect changes in capacitance due to electrical activity of the victim's heart.

18. The glove of claim 17 wherein the first and second electrodes detect changes in capacitance due to the electrical activity of the victim's heart without needing to be placed in direct contact with the victim's skin.

19. A glove for use by a rescuer when performing Cardio Pulmonary Resuscitation (CPR) compressions on a victim, the glove comprising:
at least four electrodes positioned on a ventral face of the glove at locations adapted to face the victim's chest when the glove is worn by the rescuer while applying chest compressions to the victim, the at least four electrodes adapted to detect first, second, third, and fourth ECG voltages, respectively, when the glove is worn by the rescuer and placed over the victim's chest;
an indicator positioned on a dorsal face of the glove; and
a controller adapted to determine at least first, second, third and fourth signal-to-noise ratios (SNR) of the first, second, third, and fourth ECG voltages, respectively, and to output a signal indicative of cardiac activity of the victim's heart based upon the ECG voltage with the highest SNR.

20. The glove of claim 19 wherein the controller is further adapted to output a signal indicative of cardiac activity of the victim's heart based upon the ECG voltage with the second highest SNR.

21. The glove of claim 19 wherein the indicator is a graphical display adapted to display a first graph derived from the ECG voltage with the highest SNR and a second graph derived from the ECG voltage with the second highest SNR.

22. The glove of claim 19 wherein the indicator includes a Light Emitting Diode (LED) adapted to have its illumination state changed each time the victim's heart is detected to beat.

23. The glove of claim 19 further including at least four accelerometers, each of the accelerometers positioned adjacent a corresponding one of the four electrodes, wherein the controller is further adapted to use outputs from the at least four accelerometers to remove artifacts from the first, second, third, and fourth ECG voltages that are due to movement of the rescuer when applying chest compressions to the victim.

24. The glove of claim 19 wherein a first one of the electrodes is positioned adjacent a lower palm area, a second one of the electrodes is positioned adjacent a first fingertip of the glove; and a third one of the electrodes is positioned adjacent a second fingertip of the glove.

25. The glove of claim 23 wherein the controller is further adapted to detect a depth of the chest compressions using outputs from at least one of the four accelerometers.

26. The glove of claim 21 wherein the graphical display is positioned outside of an opisthenar portion of the glove such that the rescuer is able to place his or her non-gloved hand over the opisthenar portion of the glove while applying chest compressions.

27. The glove of claim 26 wherein the graphical display is positioned adjacent a wrist area of the glove.
